(19) United States
(12) Patent Application Publication
(10) Pub. No.: US 2024/0041935 A1
(43) Pub. Date: Feb. 8, 2024

(54) PREVENTION AND TREATMENT OF REPRODUCTIVE FAILURE BY REGENERATIVE CELLS AND ADJUVANTS

(71) Applicant: CREATIVE MEDICAL TECHNOLOGIES, INC., Phoenix, AZ (US)

(72) Inventors: Thomas ICHIM, San Diego, CA (US); Amit PATEL, Salt Lake City, UT (US); Courtney BARTLETT, Niceville, FL (US)

(73) Assignee: CREATIVE MEDICAL TECHNOLOGIES, INC., Phoenix, AZ (US)

(21) Appl. No.: 18/363,642

(22) Filed: Aug. 1, 2023

Related U.S. Application Data
(60) Provisional application No. 63/395,252, filed on Aug. 4, 2022.

(51) Int. Cl. A61K 35/28; C12N 5/0775; A61K 35/16; A61K 38/18; A61K 38/30; A61K 45/06

(52) U.S. Cl. CPC A61K 35/28; C12N 5/0665; A61K 35/16; A61K 38/1808; A61K 38/30; A61K 38/1841; A61K 38/1891; A61K 45/06

(57) ABSTRACT

Disclosed are means of treatment for age related or premature reproductive failure through concurrent administration of regenerative cells together with adjuvants capable of enhancing activity of said regenerative cells. In one embodiment, the invention provides administration of autologous bone marrow mononuclear cells in the endometrium and ovarian tissue. Administration of said type of regenerative cells is combined with anti-oxidants and/or regenerative adjuvants capable of reducing inflammation and/or fibrosis, as well as providing a suitable environment for optimal regenerative function of stem cells.

### CROSS-REFERENCE TO RELATED APPLICATIONS

This application claims the benefit of priority to U.S. Provisional Application No. 63/395,252, titled ‘Prevention and Treatment of Reproductive Failure by Regenerative Cells and Adjuvants” filed Aug. 4, 2022, which is incorporated herein by reference in its entirety

### FIELD OF THE INVENTION

The teachings herein relate to the use of regenerative cells for preventing and treating reproductive failure, such as in ovarian and endometrial tissues.

### BACKGROUND OF THE INVENTION

Loss of fertility has devastating effects on the victim and family. It is known that the average age of loss of fertility in women in the U.S. is 51 years. Demographic studies have shown that it has increased from about 45 years in 1850, to approximately 51 in 1995. At the same time, however, the life expectancy of women has increased from 45 in 1850 to approximately 82 in 1998. As a result, because the life span in women has increased, almost 30% of a woman's lifetime will be infertile. The consequence of this shift is that many age-related diseases are increasing in incidence and need to be investigated. It is accepted in the academic literature that many health risks are known to be associated with menopause. There is a strong direct link between infertility and an increase in cardiovascular disease which is the leading cause of death in women over the age of 50. A number of observational studies have, provided evidence that hormone replacement therapy (HRT) reduces the risk of cardiovascular disease by about one-half. However, the HERS study recently reported that HRT in post-menopausal women did not prevent recurrent myocardial infarction. Recently, the Women's Health Initiative study conducted by the NIH reported a slight increase in cardiovascular disease-associated conditions in women taking HRT (5). Loss of fertility is the cessation of ovarian cyclicity resulting from the depletion of ovarian follicles by a natural process of attrition, known as atresia. Follicular maturation in the ovary is a dynamic series of events in which primordial follicles provide a finite pool from which preovulatory follicles are selected for development and ovulation, or are eliminated by atresia. The primordial follicle, the most immature stage of development, is formed in the ovary during fetal development. Because the oocyte is arrested in meiosis, this pool is non-regenerating after birth. In an on-going process, after puberty, follicles continually progress from the primordial to ovulatory stages. However, the vast majority do not develop to ovulation, but undergo cell death by atresia. As a result, the pool of primordial follicles gradually becomes depleted and ultimately, ovarian failure (menopause) ensues. As the pool of primordial follicles is depleted, this compromises the numbers of developing preovulatory follicles. Eventually, the reduction in pre-ovulatory follicles significantly alters ovarian steroid hormone production as a woman approaches menopause (perimenopause), resulting in a sharp decrease in circulating 17.beta.-estradiol and a concomitant rise in the gonadotropins follicle stimulating hormone (FSH) and luteinizing hormone (LH), due to loss of negative feedback from the ovary to the pituitary. Thus, following menopause, hormonal cyclicity ceases within the ovary and it secretes primarily androgens in a hypergonadotropic environment. Because 17.beta.-estradiol is assumed to afford protection in premenopausal women against health risks, such as cardiovascular disease, skeletal problems, and brain dysfunction, the loss of 17.beta.-estradiol in menopause is thought to contribute to most menopause-associated disorders. The invention seeks to provide means of overcoming reproductive failure through stimulation of regeneration in ovarian and endometrial tissues.

### SUMMARY

Preferred embodiments include methods for treatment of infertility comprising the steps of: a) obtaining a patient suffering from infertility, wherein said cause of infertility is associated with abnormalities of hormone production and/or responsiveness to said hormones; b) administering a regenerative cell population in a manner to stimulate ovarian functionality; c) administering a regenerative cell population in a manner to stimulate ovarian functionality; d) optionally providing prior to and/or concurrently with, and/or subsequently to, an agent or plurality of agents which alter the host in order to enhance ability of administered regenerative cells to provide a therapeutic benefit; and e) optionally providing a mitogenic and/or stem cell activator agent.

Preferred methods include embodiments wherein said infertility is as a result of premature ovarian failure.

Preferred methods include embodiments wherein said infertility is as a result of premature endometrial failure.

Preferred methods include embodiments wherein said infertility is as a result of premature uterine failure.

Preferred methods include embodiments wherein said infertility is as a result of age-associated ovarian failure.

Preferred methods include embodiments wherein said infertility is as a result of age-associated endometrial failure.

Preferred methods include embodiments wherein said infertility is as a result of age-associated uterine failure.

Preferred methods include embodiments wherein said hormonal imbalance is overproduction of follicle stimulating hormone.

Preferred methods include embodiments wherein said hormonal imbalance is underproduction of estrogen.

Preferred methods include embodiments wherein said ovarian and/or endometrial deficiency is mediated by fibrosis.

Preferred methods include embodiments wherein said regenerative cell is plastic adherent.

Preferred methods include embodiments wherein said plastic adherent cell is a mesenchymal stem cell.

Preferred methods include embodiments wherein said mesenchymal stem cells are derived from fluids.

Preferred methods include embodiments wherein said fluid is plasma.

Preferred methods include embodiments wherein said fluid is cerebral spinal fluid.

Preferred methods include embodiments wherein said fluid is urine.

Preferred methods include embodiments wherein said fluid is seminal fluid.

Preferred methods include embodiments wherein said mesenchymal stem cells are derived from tissues.

Preferred methods include embodiments wherein said tissue derived mesenchymal stem cells are selected from a group comprising of: a) bone marrow; b) perivascular tissue; c) adipose tissue; d) placental tissue; e) amniotic membrane; f) omentum; g) tooth; h) umbilical cord tissue; i) fallopian tube tissue; j) hepatic tissue; k) renal tissue; l) cardiac tissue; m) tonsillar tissue; n) testicular tissue; o) ovarian tissue; p) neuronal tissue; q) auricular tissue; r) colonic tissue; s) submucosal tissue; t) hair follicle tissue; u) pancreatic tissue; v) skeletal muscle tissue; and w) subepithelial umbilical cord tissue.

Preferred methods include embodiments wherein said tissue derived mesenchymal stem cells are isolated from tissues containing cells selected from a group of cells comprising of: endothelial cells, epithelial cells, dermal cells, endodermal cells, mesodermal cells, fibroblasts, osteocytes, chondrocytes, natural killer cells, dendritic cells, hepatic cells, pancreatic cells, stromal cells, and salivary gland mucous cells.

Preferred methods include embodiments wherein said mesenchymal stem cells are plastic adherent and xeno-free.

Preferred methods include embodiments wherein said mesenchymal stem cells express a marker selected from a group comprising of: a) CD73; b) CD90; and c) CD105.

Preferred methods include embodiments wherein said mesenchymal stem cells are derived from umbilical cord tissue and lack expression of a marker selected from a group comprising of: a) CD14; b) CD45; and c) CD34.

Preferred methods include embodiments wherein said mesenchymal stem cells from umbilical cord tissue express markers selected from a group comprising of; a) oxidized low density lipoprotein receptor 1; b) chemokine receptor ligand 3; and c) granulocyte chemotactic protein.

Preferred methods include embodiments wherein said mesenchymal stem cells from umbilical cord tissue do not express markers selected from a group comprising of: a) CD117; b) CD31; c) CD34; and d) CD45.

Preferred methods include embodiments wherein said mesenchymal stem cells from umbilical cord tissue express, relative to a human fibroblast, increased levels of interleukin 8 and reticulon 1

Preferred methods include embodiments wherein said mesenchymal stem cells from umbilical cord tissue have the potential to differentiate into cells of at least a skeletal muscle, vascular smooth muscle, pericyte or vascular endothelium phenotype.

Preferred methods include embodiments wherein said mesenchymal stem cells from umbilical cord tissue express markers selected from a group comprising of: a) CD10; b) CD13; c) CD44; d) CD73; and e) CD90.

Preferred methods include embodiments wherein said umbilical cord tissue mesenchymal stem cell is an isolated umbilical cord tissue cell isolated from umbilical cord tissue substantially free of blood that is capable of self-renewal and expansion in culture for 80 doublings.

Preferred methods include embodiments wherein said umbilical cord tissue mesenchymal stem cells has the potential to differentiate into cells of other phenotypes.

Preferred methods include embodiments wherein said other phenotypes comprise: a) osteocytic; b) adipogenic; and c) chondrogenic differentiation.

Preferred methods include embodiments wherein said cord tissue derived mesenchymal stem cells can undergo at least 20 doublings in culture.

Preferred methods include embodiments wherein said cord tissue derived mesenchymal stem cell maintains a normal karyotype upon passaging

Preferred methods include embodiments wherein said cord tissue derived mesenchymal stem cell expresses a marker selected from a group of markers comprised of: a) CD10; b) CD13; c) CD44; d) CD73; e) CD90; f) PDGFr-alpha; g) PD-L2; and h) HLA-A, -B, -C.

Preferred methods include embodiments wherein said cord tissue mesenchymal stem cells does not express one or more markers selected from a group comprising of; a) CD31; b) CD34; c) CD45; d) CD80; e) CD86; f) CD117; g) CD141; h) CD178; i) B7-H2; j) HLA-G and k) HLA-DR, -DP, -DQ.

Preferred methods include embodiments wherein said umbilical cord tissue-derived cell secretes factors selected from a group comprising of: a) MCP-1; b) MIPlbeta; c) IL-6; d) IL-8; e) GCP-2; f) HGF; g) KGF; h) FGF; i) HB-EGF; j) BDNF; k) TPO; l) RANTES; and m) TIMP1.

Preferred methods include embodiments wherein said umbilical cord tissue derived cells express markers selected from a group comprising of: a) TRA1-60; b) TRA1-81; c) SSEA3; d) SSEA4; and e) NANOG.

Preferred methods include embodiments wherein said umbilical cord tissue-derived cells are positive for alkaline phosphatase staining.

Preferred methods include embodiments in which the patient is prepared for administration of regenerative cells by evoking a regenerative state in the ovary and/or endometrium.

Preferred methods include embodiments wherein said regenerative state is evoked by administration of extracorporeal shock waves.

Preferred methods include embodiments wherein said extracorporeal shock wave generates a flux density higher than 0.3 mJ/mm.sup.2.

Preferred methods include embodiments wherein said extracorporeal shock wave generates a flux density less than 0.3 mJ/mm.sup.2 and higher than 0.1 mJ/mm.sup.2.

Preferred methods include embodiments wherein said extracorporeal shock wave generates a flux density less than 0.1 mJ/mm.sup.2.

Preferred methods include embodiments wherein said extracorporeal shock wave is administered through a frequency of the shots (1-15 Hz) and the number of shocks per one session (500-50,000).

Preferred methods include embodiments wherein said extracorporeal shock wave is administered at an energy of 1-1000 joules.

Preferred embodiments include methods wherein stimulation of ovarian function in a patient suffering from ovarian failure further comprises the steps of: a) obtaining peripheral blood; b) isolating platelet rich plasma, and/or platelet lysate (fresh, frozen and lyophilized); c) quantifying growth factor content of said platelet rich plasma and/or platelet lysate; d) optionally concentrating said growth factors from said platelet rich plasma and/or platelet lysate derived growth factors; and e) administering said growth factors locally into ovarian tissue in a patient in need of treatment.

Preferred methods include embodiments wherein said growth factor associated with platelet rich plasma and/or platelet lysate is EGF-1.

Preferred methods include embodiments wherein said growth factor associated with platelet rich plasma and/or platelet lysate is IGF-1.

Preferred methods include embodiments wherein said growth factor associated with platelet rich plasma and/or platelet lysate is HGF-1.

Preferred methods include embodiments wherein said growth factor associated with platelet rich plasma and/or platelet lysate is VEGF.

Preferred methods include embodiments wherein said growth factor associated with platelet rich plasma and/or platelet lysate is FGF-1.

Preferred methods include embodiments wherein said growth factor associated with platelet rich plasma and/or platelet lysate is FGF-2.

Preferred methods include embodiments wherein said growth factor associated with platelet rich plasma and/or platelet lysate is FGF-5.

Preferred methods include embodiments wherein said growth factor associated with platelet rich plasma and/or platelet lysate is PDGF-1.

Preferred methods include embodiments wherein said growth factor associated with platelet rich plasma and/or platelet lysate is activin.

Preferred methods include embodiments wherein said growth factor associated with platelet rich plasma and/or platelet lysate is TGF-alpha.

Preferred methods include embodiments wherein said growth factor associated with platelet rich plasma and/or platelet lysate is TGF-beta.

Preferred methods include embodiments wherein said growth factor associated with platelet rich plasma and/or platelet lysate is interleukin-10.

Preferred methods include embodiments wherein said growth factor associated with platelet rich plasma and/or platelet lysate is interleukin-4.

Preferred methods include embodiments wherein said growth factor associated with platelet rich plasma and/or platelet lysate is interleukin-13.

Preferred methods include embodiments wherein said growth factor associated with platelet rich plasma and/or platelet lysate is interleukin-27.

Preferred methods include embodiments wherein said growth factor associated with platelet rich plasma and/or platelet lysate is interleukin-20.

Preferred methods include embodiments wherein said growth factor associated with platelet rich plasma and/or platelet lysate is interleukin-1 receptor antagonist.

Preferred methods include embodiments wherein said growth factor associated with platelet rich plasma and/or platelet lysate is chorionic gonadotropin.

Preferred methods include embodiments wherein said growth factor associated with platelet rich plasma and/or platelet lysate is HLA-G.

Preferred methods include embodiments wherein said growth factor associated with platelet rich plasma and/or platelet lysate is inhibin.

Preferred methods include embodiments wherein said growth factor associated with platelet rich plasma and/or platelet lysate is beta microglobulin.

Preferred methods include embodiments wherein said growth factor associated with platelet rich plasma and/or platelet lysate is angiopoietin.

Preferred methods include embodiments wherein said growth factor associated with platelet rich plasma and/or platelet lysate is G-CSF.

Preferred methods include embodiments wherein said growth factor associated with platelet rich plasma and/or platelet lysate is M-CSF.

Preferred methods include embodiments wherein said growth factor associated with platelet rich plasma and/or platelet lysate is GM-CSF.

Preferred methods include embodiments wherein said growth factor associated with platelet rich plasma and/or platelet lysate is endostatin.

Preferred methods include embodiments wherein said growth factor associated with platelet rich plasma and/or platelet lysate is plasminogen.

Preferred methods include embodiments wherein said growth factors are exosomes.

Preferred methods include embodiments wherein said growth factors are microvesicles.

Preferred methods include embodiments wherein said growth factors are subcellular fragments.

Preferred methods include embodiments wherein said administration into ovarian tissue is performed at a concentration and frequency sufficient to induce differentiation of oocyte progenitor cells.

Preferred methods include embodiments wherein said administration into ovarian tissue is performed at a concentration and frequency sufficient to induce reduction of fibrotic damage to said ovarian tissue.

Preferred methods include embodiments wherein said administration into ovarian tissue is performed at a concentration and frequency sufficient to induce reduction of IL-17 in said ovarian tissue.

Preferred methods include embodiments wherein increasing fertility is achieved through the steps comprising of: a) extracting an autologous population of regenerative cells; b) treating said autologous population of regenerative cells with platelet rich plasma, and/or platelet lysate at a concentration and duration sufficient to induce type 2 cytokine polarization; and c) administering said treated regenerative cells into a patient in need of treatment.

Preferred methods include embodiments wherein said autologous regenerative cells are bone marrow mononuclear cells/aspirate.

Preferred methods include embodiments wherein said autologous regenerative cells are bone marrow mononuclear cells/aspirate contain CD34 cells.

Preferred methods include embodiments wherein said autologous regenerative cells are bone marrow mononuclear cells/aspirate contain approximately 0.01% to 5% CD34 cells.

Preferred methods include embodiments wherein said autologous regenerative cells are bone marrow mononuclear cells/aspirate contain CD133 cells.

Preferred methods include embodiments wherein said autologous regenerative cells are bone marrow mononuclear cells contain/aspirate CD14 cells.

Preferred methods include embodiments wherein said autologous regenerative cells are bone marrow mononuclear cells contain/aspirate CD56 cells.

Preferred methods include embodiments wherein said autologous regenerative cells are bone marrow mononuclear cells/aspirate contain c-kit expressing cells.

Preferred methods include embodiments wherein said autologous regenerative cells are adipose stromal vascular fraction cells.

Preferred methods include embodiments wherein said autologous regenerative cells are peripheral blood mononuclear cells.

Preferred methods include embodiments wherein said peripheral blood mononuclear cells are collected subsequent to administration of a mobilization means.

Preferred methods include embodiments wherein said mobilization means is G-CSF administration.

Preferred methods include embodiments wherein said mobilization means is Mozibil administration.

Preferred methods include embodiments wherein said mobilization means is FLR-3L administration.

Preferred methods include embodiments wherein agents possessing anti-inflammatory properties are selected from a group comprising of: More specifically, anti-inflammatory agents may comprise one or more of, e.g., anti-TNF-α, lysophylline, alpha 1-antitrypsin (AAT), interleukin-10 (IL-10), pentoxyfilline, COX-2 inhibitors, 21-acetoxypregnenolone, alclometasone, algestone, amcinonide, beclomethasone, betamethasone, budesonide, chloroprednisone, clobetasol, clobetasone, clocortolone, cloprednol, corticosterone, cortisone, cortivazol, deflazacort, desonide, desoximetasone, dexamethasone, diflorasone, diflucortolone, difluprednate, enoxolone, fluazacort, flucloronide, flumethasone, flunisolide, fluocinolone acetonide, fluocinonide, fluocortin butyl, fluocortolone, fluorometholone, fluperolone acetate, fluprednidene acetate, fluprednisolone, flurandrenolide, fluticasone propionate, formocortal, halcinonide, halobetasol propionate, halometasone, halopredone acetate, hydrocortamate, hydrocortisone, loteprednol etabonate, mazipredone, medrysone, meprednisone, methylprednisolone, mometasone furoate, paramethasone, prednicarbate, prednisolone, prednisolone 25-diethylamino-acetate, prednisolone sodium phosphate, prednisone, prednival, prednylidene, rimexolone, tixocortol, triamcinolone, triamcinolone acetonide, triamcinolone benetonide, triamcinolone hexacetonide, aminoarylcarboxylic acid derivatives (e.g., enfenamic acid, etofenamate, flufenamic acid, isonixin, meclofenamic acid, mefenamic acid, niflumic acid, talniflumate, terofenamate, tolfenamic acid), arylacetic acid derivatives (e.g., aceclofenac, acemetacin, alclofenac, amfenac, amtolmetin guacil, bromfenac, bufexamac, cinmetacin, clopirac, diclofenac sodium, etodolac, felbinac, fenclozic acid, fentiazac, glucametacin, ibufenac, indomethacin, isofezolac, isoxepac, lonazolac, metiazinic acid, mofezolac, oxametacine, pirazolac, proglumetacin, sulindac, tiaramide, tolmetin, tropesin, zomepirac), arylbutyric acid derivatives (e.g., bumadizon, butibufen, fenbufen, xenbucin), arylcarboxylic acids (e.g., clidanac, ketorolac, tinoridine), arylpropionic acid derivatives (eg., alminoprofen, benoxaprofen, bermoprofen, bucloxic acid, carprofen, fenoprofen, flunoxaprofen, flurbiprofen, ibuprofen, ibuproxam, indoprofen, ketoprofen, loxoprofen, naproxen, oxaprozin, piketoprolen, pirprofen, pranoprofen, protizinic acid, suprofen, tiaprofenic acid, ximoprofen, zaltoprofen), pyrazoles (e.g., difenamizole, epirizole), pyrazolones (e.g., apazone, benzpiperylon, feprazone, mofebutazone, morazone, oxyphenbutazone, phenylbutazone, pipebuzone, propyphenazone, ramifenazone, suxibuzone, thiazolinobutazone), salicylic acid derivatives (e.g., acetaminosalol, aspirin, benorylate, bromosaligenin, calcium acetylsalicylate, diflunisal, etersalate, fendosal, gentisic acid, glycol salicylate, imidazole salicylate, lysine acetylsalicylate, mesalamine, morpholine salicylate, 1-naphthyl salicylate, olsalazine, parsalmide, phenyl acetylsalicylate, phenyl salicylate, salacetamide, salicylamide o-acetic acid, salicylsulfuric acid, salsalate, sulfasalazine), thiazinecarboxamides (e.g., ampiroxicam, droxicam, isoxicam, lornoxicam, piroxicam, tenoxicam), epsilon.-acetamidocaproic acid, s-adenosylmethionine, 3-amino-4-hydroxybutyric .acid, amixetrine, bendazac, benzydamine, α-bisabolol, bucolome, difenpiramide, ditazol, emorfazone, fepradinol, guaiazulene, nabumetone, nimesulide, oxaceprol, paranyline, perisoxal, proquazone, superoxide dismutase, tenidap, zileuton, candelilla wax, alpha bisabolol, aloe vera, Manjistha, Guggal, kola extract, chamomile, sea whip extract, glycyrrhetic acid, glycyrrhizic acid, oil soluble licorice extract, monoammonium glycyrrhizinate, monopotassium glycyrrhizinate, dipotassium glycyrrhizinate, 1-beta-glycyrrhetic acid, stearyl glycyrrhetinate, and 3-stearyloxy-glycyrrhetinic acid.

Preferred methods include embodiments wherein the autologous regenerative cell is combined with an anti-oxidant to increase therapeutic benefit and cell survival from beta carotene, Vitamin E, anthocyanins, selenium, catechins, lutein, lycopene, n-acetylcysteine, ascorbic acid, glutathione, vitamin k3, resveratrol, α lipoic acid, quercetin, kaempferol, myricetin, apigenin, luteolin, curcumin, caffeic acid.

Preferred methods include embodiments wherein the autologous regenerative cell is combined with fertility hormones Gonal-F, Follistim, Bravelle, Menopur, Repronex, Estrace, Clomid, serophene, follicle stimulation hormone, luteinizing hormone, human chorionic gonadotrophin, estrogen, GnRH antagonist, progesterone, gonadotropin-releasing hormone, Human menopausal gonadotropin, Metrodin, Pergonal, Factrel, Lutrepulse, Lupron, Synarel, Zoladex, Antagon, Cetrotide, Novarel, Ovidrel, Pregnyl, Profasi, Antagon, Dostinex, Parlodel, or Fertinex.

### DETAILED DESCRIPTION OF THE INVENTION

In the broadest embodiment, the invention teaches the utilization of regenerative cells together with agents capable of altering the ovarian and endometrial environments to enhance therapeutic activities of said regenerative cells. According to the invention, the utilization of agents that suppress NF-kappa B activation and concentration-dependently increases DPPH free radical scavenging activity so as to have excellent antioxidant activity, inhibits apoptosis of stem cells injected into the ovary and endometrium, and activate a PI3K/Akt signaling pathway so as to have a significant protective effect against endometrial and ovarian loss. Therefore, infertility or subfertility treatment or prevention, premature ovarian failure, a menopausal disorder in the premenopausal period, and the like can be considered application examples related thereto.

In some embodiments, a population of stem cells are utilized to induce regeneration of ovarian and endometrial tissue. In some embodiments, administration of “resilient” mesenchymal stem cells, which have been pulsed by various means such as hypoxia, is associated with endowment of expression of the initerleukin-3 receptor on ovarian tissue. For the practice of the invention, MSC are used to induce dedifferentiation or rejuvenation of ovarian and/or endometrial tissues. In some embodiments additional antioxidants are utilized in order to enhance therapeutic effects.

As used herein, the terms “or” and “and/or” are utilized to describe multiple components in combination or exclusive of one another. For example, “x, y, and/or z” can refer to “x” alone, “y” alone, “z” alone, “x, y, and z,” “(x and y) or z,” “x or (y and z),” or “x or y or z.” It is specifically contemplated that x, y, or z may be specifically excluded from an embodiment.

Throughout this application, the term “about” is used according to its plain and ordinary meaning in the area of cell and molecular biology to indicate that a value includes the standard deviation of error for the device or method being employed to determine the value.

Chemical Modification: As used herein, “chemical modification” refers to the process wherein a chemical or biochemical is used to induce genomic changes in the donor cell, or nucleus thereof, that allow the donor cell, or nucleus thereof, to be responsive during maturation and receptive to the host cell cytoplasm.

Committed: As used herein, “committed” refers to cells which are considered to be permanently committed to a specific function. Committed cells are also referred to as “terminally differentiated cells.”

Cytoplast Extract Modification: As used herein, “cytoplast extract modification” refers to the process wherein a cellular extract consisting of the cytoplasmic contents of a cell are used to induce genomic changes in the donor cell, or nucleus thereof, that allow the donor cell, or nucleus thereof, to be responsive during maturation and receptive to the host cell cytoplasm.

Dedifferentiation: As used herein, “dedifferentiation” refers to loss of specialization in form or function. In cells, dedifferentiation leads to an a less committed cell.

Differentiation: As used herein, “differentiation” refers to the adaptation of cells for a particular form or function. In cells, differentiation leads to a more committed cell.

Donor Cell: As used herein, “donor cell” refers to any diploid (2N) cell derived from a pre-embryonic, embryonic, fetal, or post-natal multi-cellular organism or a primordial sex cell which contributes its nuclear genetic material to the hybrid stem cell. The donor cell is not limited to those cells that are terminally differentiated or cells in the process of differentiation. For the purposes of this invention, donor cell refers to both the entire cell or the nucleus alone.

Donor Cell Preparation: As used herein, “donor cell preparation” refers to the process wherein the donor cell, or nucleus thereof, is prepared to undergo maturation or prepared to be receptive to a host cell cytoplasm and/or responsive within a post-natal environment.

Germ Cell: As used herein, “germ cell” refers to a reproductive cell such as a spermatocyte or an oocyte, or a cell that will develop into a reproductive cell.

Host Cell: As used herein, “host cell” refers to any multipotent stem cell derived from a pre-embryonic, embryonic, fetal, or post-natal multicellular organism that contributes the cytoplasm to a hybrid stem cell.

Host Cell Preparation: As used herein, “host cell preparation” refers to the process wherein the host cell is enucleated.

Hybrid Stem Cell: As used herein, “hybrid stem cell” refers to any cell that is multipotent and is derived from an enucleated host cell and a donor cell, or nucleus thereof, of a multicellular organism. Hybrid stem cells are further disclosed in co-pending U.S. patent application Ser. No. 10/864,788.

Karyoplast Extract Modification: As used herein, “karyoplast extract modification” refers to the process wherein a cellular extract consisting of the nuclear contents of a cell, lacking the DNA, are used to induce genomic changes in the donor cell, or nucleus thereof, that allow the donor cell, or nucleus thereof, to be responsive during maturation or receptive to the host cell cytoplasm.

Maturation: As used herein, “maturation” refers to a process of coordinated steps either forward or backward in the differentiation pathway and can refer to both differentiation or de-differentiation. As used herein, maturation is synonymous with the terms develop or development when applied to the process described herein.

“Mesenchymal stem cell” or “MSC” in some embodiments refers to cells that are (1) adherent to plastic, (2) express CD73, CD90, and CD105 antigens, while being CD14, CD34, CD45, and HLA-DR negative, and (3) possess ability to differentiate to osteogenic, chondrogenic and adipogenic lineage. Other cells possessing mesenchymal-like properties are included within the definition of “mesenchymal stem cell”, with the condition that said cells possess at least one of the following: a) regenerative activity; b) production of growth factors; c) ability to induce a healing response, either directly, or through elicitation of endogenous host repair mechanisms. As used herein, “mesenchymal stromal cell” or mesenchymal stem cell can be used interchangeably. Said MSC can be derived from any tissue including, but not limited to, bone marrow, adipose tissue, amniotic fluid, endometrium, trophoblast-derived tissues, cord blood, Wharton jelly, placenta, amniotic tissue, derived from pluripotent stem cells, and tooth. In some definitions of “MSC”, said cells include cells that are CD34 positive upon initial isolation from tissue but are similar to cells described about phenotypically and functionally. As used herein, “MSC” may include cells that are isolated from tissues using cell surface markers selected from the list comprised of NGF-R, PDGF-R, EGF-R, IGF-R, CD29, CD49a, CD56, CD63, CD73, CD105, CD106, CD140b, CD146, CD271, MSCA-1, SSEA4, STRO-1 and STRO-3 or any combination thereof, and satisfy the ISCT criteria either before or after expansion. Furthermore, as used herein, in some contexts, “MSC” includes cells described in the literature as bone marrow stromal stem cells (BMSSC), marrow-isolated adult multipotent inducible cells (MIAMI) cells, multipotent adult progenitor cells (MAPC), mesenchymal adult stem cells (MASCS), MultiStem®, Prochymal®, remestemcel-L, Mesenchymal Precursor Cells (MPCs), Dental Pulp Stem Cells (DPSCs), PLX cells, PLX-PAD, AlloStem®, Astrostem®, Ixmyelocel-T, MSC-NTF, NurOwn™, Stemedyne™-MSC, Stempeucel®, StempeucelCLI, StempeucelOA, HiQCell, Hearticellgram-AMI, Revascor®, Cardiorel®, Cartistem®, Pneumostem®, Promostem®, Homeo-GH, AC607, PDA001, SB623, CX601, AC607, Endometrial Regenerative Cells (ERC), adipose-derived stem and regenerative cells (ADRCs). the population of umbilical cord cells has an ability to undergo cell division in less than 36 hours in a growth medium. In some embodiments, the population of cells, the population of umbilical cord cells has an ability to proliferate at a rate of 0.9-1.2 doublings per 36 hours in growth media. In some embodiments, the population of cells, the population of umbilical cord cells has an ability to proliferate at a rate of 0.9, 1.0, 1.1, or 1.2 doublings per 36 hours in growth media. The population of cells, population of umbilical cord cells may produce exosomes capable of inducing more than 50% proliferation when the exosomes are cultured with human umbilical cord endothelial cells. The induction of proliferation may occur when the exosomes are cultured with the human umbilical cord endothelial cells at a concentration of 10, 20, 30, 40, 50, 70, 80, 90, 100, 110, 120, 130, 140, 150, 160, 170, 180, 190, 200, or more exosomes per cell.

In some embodiments, a population of cells, including a population of umbilical cells alone, are administered to an individual, including an individual having and acute or chronic pathology, wherein the population of cells may be administered via any suitable route, including as non-limiting examples, intramuscularly and/or intravenously.

Multipotent Adult Progenitor Cells: As used herein, “multipotent adult progenitor cells” refers to multipotent cells isolated from the bone marrow which have the potential to differentiate into mesenchymal, endothelial and endodermal lineage cells.

Pluripotent: As used herein, “pluripotent” refers to cells that can give rise to any cell type except the cells of the placenta or other supporting cells of the uterus.

Primordial Sex Cell: As used herein, “primordial sex cell” refers to any diploid cell that is derived from the male or female mature or developing gonad, is able to generate cells that propagate a species and contains a diploid genomic state. Primordial sex cells can be quiescent or actively dividing. These cells include male gonocytes, female gonocytes, spermatogonial stem cells, ovarian stem cells, oogonia, type-A spermatogonia, Type-B spermatogonia. Also known as germ-line stem cells.

Primordial Germ Cell: As used herein, “primordial germ cell” refers to cells present in early embryogenesis that are destined to become germ cells.

Reprogramming: As used herein “reprogramming” refers to the resetting of the genetic program of a cell such that the cell exhibits pluripotency and has the potential to produce a fully developed organism.

Responsive: As used herein, “responsive” refers to the condition of a cell, or group of cells, wherein they are susceptible to and can function accordingly within a cellular environment. Responsive cells are capable of responding to and functioning in a particular cellular environment, tissue, organ and/or organ system.

Somatic Stem Cells: As used herein, “somatic stem cells” refers to diploid multipotent or pluripotent stem cells. Somatic stem cells are not totipotent stem cells. Stem cells are primitive cells that give rise to other types of cells. Also called progenitor cells, there are several kinds of stem cells. Totipotent cells are considered the “master” cells of the body because they contain all the genetic information needed to create all the cells of the body plus the placenta, which nourishes the human embryo. Human cells have this totipotent capacity only during the first few divisions of a fertilized egg. After three to four divisions of totipotent cells, there follows a series of stages in which the cells become increasingly specialized. The next stage of division results in pluripotent cells, which are highly versatile and can give rise to any cell type except the cells of the placenta or other supporting tissues of the uterus. At the next stage, cells become multipotent, meaning they can give rise to several other cell types, but those types are limited in number. An example of multipotent cells is hematopoietic cells—blood cells that can develop into several types of blood cells, but cannot develop into brain cells. At the end of the long chain of cell divisions that make up the embryo are “terminally differentiated” cells—cells that are considered to be permanently committed to a specific function.

Whole Cell Extract Modification: As used herein, “whole cell extract modification” refers to the process wherein a cellular extract consisting of the cytoplasmic and nuclear contents of a cell are used to induce genomic changes in the donor cell, or nucleus thereof, that allow the donor cell, or nucleus thereof, to be responsive during maturation and receptive to the host cell cytoplasm.

In one embodiment the invention teaches phenotypically defined MSC which can be isolated from the Wharton's jelly of umbilical cord segments and defined morphologically and by cell surface markers. By dissecting out the veins and arteries of cord segments and exposing the Wharton's jelly, the cells of invention, of one embodiment of the invention, may be obtained. An approximately 1-5 cm cord segment is placed in collagenase solution (1 mg/ml, Sigma) for approximately 18 hours at room temperature. After incubation, the remaining tissue is removed and the cell suspension is diluted with PBS into two 50 ml tubes and centrifuged. Cells are then washed in PBS and counted using hematocytometer. 5-20.times.10.sup.6 cells were then plated in a 6 cm tissue culture plate in low-glucose DMEM (Gibco) with 10% FBS (Hyclone), 2 mM L-Glutamine (Gibco), 100 U/ml penicillin/100 ug/ml streptomycin/0.025 ug/ml amphotericin B (Gibco). At this step of the purification process, cells are exposed to hypoxia. The amount of hypoxia needed is the sufficient amount to induce activation of HIF-1 alpha. In one embodiment cells are cultured for 24 hours at 2% oxygen. After 48 hours, cells are washed with PBS and given fresh media. Cells were given new media twice weekly. After 7 days, cells are approximately 70-80% confluent and are passed using HyQTase (Hyclone) into a 10 cm plate. Cells are then regularly passed 1:2 every 7 days or upon reaching 80% confluence.

In another embodiment of the invention, biologically useful stem cells are disclosed, of the mesenchymal or related lineages, which are therapeutically reprogrammed cells having minimal oxidative damage and telomere lengths that compare favorably with the telomere lengths of undamaged, pre-natal or embryonic stem cells (that is, the therapeutically reprogrammed cells of the present invention possess near prime physiological state genomes). Moreover, the therapeutically reprogrammed cells of the present invention are immunologically privileged and therefore suitable for therapeutic applications. Additional methods of the present invention provide for the generation of hybrid stem cells. Furthermore, the present invention includes related methods for maturing stem cells made in accordance with the teachings of the present invention into specific host tissues. For use in the current invention, the practitioner is thought that ontogeny of mammalian development provides a central role for stem cells. Early in embryogenesis, cells from the proximal epiblast destined to become germ cells (primordial germ cells) migrate along the genital ridge. These cells express high levels of alkaline phosphatase as well as expressing the transcription factor Oct4. Upon migration and colonization of the genital ridge, the primordial germ cells undergo differentiation into male or female germ cell precursors (primordial sex cells). For the purpose of this invention disclosure, only male primordial sex cells (PSC) will be discussed, but the qualities and properties of male and female primordial sex cells are equivalent and no limitations are implied. During male primordial sex cell development, the primordial stem cells become closely associated with precursor sertoli cells leading to the beginning of the formation of the seminiferous cords. When the primordial germ cells are enclosed in the seminiferous cords, they differentiate into gonocytes that are mitotically quiescent. These gonocytes divide for a few days followed by arrest at G0/G1 phase of the cell cycle. In mice and rats these gonocytes resume division within a few days after birth to generate spermatogonial stem cells and eventually undergo differentiation and meiosis related to spermatogenesis. It is known that embryonic stem cells are cells derived from the inner cell mass of the pre-implantation blastocyst-stage embryo and have the greatest differentiation potential, being capable of giving rise to cells found in all three germ layers of the embryo proper. From a practical standpoint, embryonic stem cells are an artifact of cell culture since, in their natural epiblast environment, they only exist transiently during embryogenesis. Manipulation of embryonic stem cells in vitro has led to the generation and differentiation of a wide range of cell types, including cardiomyocytes, hematopoietic cells, endothelial cells, nerves, skeletal muscle, chondrocytes, adipocytes, liver and pancreatic islets. Growing embryonic stem cells in co-culture with mature cells can influence and initiate the differentiation of the embryonic stem cells to a particular lineage. Maturation is a process of coordinated steps either forward or backward in the differentiation pathway and can refer to both differentiation and/or dedifferentiation. In one example of the maturation process, a cell, or group of cells, interacts with its cellular environment during embryogenesis and organogenesis. As maturation progresses, cells begin to form niches and these niches, or microenvironments, house stem cells that direct and regulate organogenesis. At the time of birth, maturation has progressed such that cells and appropriate cellular niches are present for the organism to function and survive post-natally. Developmental processes are highly conserved amongst the different species allowing maturation or differentiation systems from one mammalian species to be extended to other mammalian species in the laboratory. During the lifetime of an organism, the cellular composition of the organs and organs systems are exposed to a wide range of intrinsic and extrinsic factors that induce cellular or genomic damage. Ultraviolet light not only has an effect on normal skin cells but also on the skin stem cell population. Chemotherapeutic drugs used to treat cancer have a devastating effect on hematopoietic stem cells. Reactive oxygen species, which are the byproducts of cellular metabolism, are intrinsic factors that compromises the genomic integrity of the cell. In all organs or organ systems, cells are continuously being replaced from stem cell populations. However, as an organism ages, cellular damage accumulates in these stem cell populations. If the damage is inheritable, such as genomic mutations, then all progeny will be effected and thus compromised. A single stem cell clone can contribute to generations of lineages such as lymphoid and myeloid cells for more than a year and therefore have the potential to spread mutations if the stem cell is damaged. The body responds to a compromised stem cell by inducing apoptosis thereby removing it from the pool and preventing potentially dysfunctional or tumorigenic properties. Apoptosis removes compromised cells from the population, but it also decreases the number of stem cells that are available for the future. Therefore, as an organism ages, the number of stem cells decrease. In addition to the loss of the stem cell pool, there is evidence that aging decreases the efficiency of the homing mechanism of stem cells. Telomeres are the physical ends of chromosomes that contain highly conserved, tandemly repeated DNA sequences. Telomeres are involved in the replication and stability of linear DNA molecules and serve as counting mechanism in cells; with each round of cell division the length of the telomeres shortens and at a pre-determined threshold, a signal is activated to initiate cellular senescence. Stem cells and somatic cells produce telomerase, which inhibits shortening of telomeres, but their telomeres still progressively shorten during aging and cellular stress. In one teaching, or embodiment, of the invention, therapeutically reprogrammed cells, in some embodiments mesenchymal stem cells, are provided. Therapeutic reprogramming refers to a maturation process wherein a stem cell is exposed to stimulatory factors according the teachings of the present invention to yield enhanced therapeutic activity. In some embodiments, enhancement of therapeutic activity may be increase proliferation, in other embodiments, it may be enhanced chemotaxis. Other therapeutic characteristics include ability to under resistance to apoptosis, ability to overcome senescence, ability to differentiate into a variety of different cell types effectively, and ability to secrete therapeutic growth factors which enhance viability/activity, of endogenous stem cells. In order to induce therapeutic reprogramming of cells, in some cases, as disclosed herein, of wharton's jelly originating cells, the invention teaches the utilization of stimulatory factors, including without limitation, chemicals, biochemicals and cellular extracts to change the epigenetic programming of cells. These stimulatory factors induce, among other results, genomic methylation changes in the donor DNA. Embodiments of the present invention include methods for preparing cellular extracts from whole cells, cytoplasts, and karyoplasts, although other types of cellular extracts are contemplated as being within the scope of the present invention. In a non-limiting example, the cellular extracts of the present invention are prepared from stem cells, specifically embryonic stem cells. Donor cells are incubated with the chemicals, biochemicals or cellular extracts for defined periods of time, in a non-limiting example for approximately one hour to approximately two hours, and those reprogrammed cells that express embryonic stem cell markers, such as Oct4, after a culture period are then ready for transplantation, cryopreservation or further maturation. In another embodiment of the present invention, hybrid stem cells are provided which can be used for cellular regenerative/reparative therapy. The hybrid stem cells of the present invention are pluripotent and customized for the intended recipient so that they are immunologically compatible with the recipient. Hybrid stem cells are a fusion product between a donor cell, or nucleus thereof, and a host cell. Typically, the fusion occurs between a donor nucleus and an enucleated host cell. The donor cell can be any diploid cell, including but not limited to, cells from pre-embryos, embryos, fetuses and post-natal organisms. More specifically, the donor cell can be a primordial sex cell, including but not limited to, oogonium or differentiated or undifferentiated spermatogonium, or an embryonic stem cell. Other non-limiting examples of donor cells are therapeutically reprogrammed cells, embryonic stem cells, fetal stem cells and multipotent adult progenitor cells. Preferably the donor cell has the phenotype of the intended recipient. The host cell can be isolated from tissues including, but not limited to, pre-embryos, embryos, fetuses and post-natal organisms and more specifically can include, but is not limited to, embryonic stem cells, fetal stem cells, multipotent adult progenitor cells and adipose-derived stem cells. In a non-limiting example, cultured cell lines can be used as donor cells. The donor and host cells can be from the same individual or different individuals. In one embodiment of the present invention, lymphocytes are used as donor cells and a two-step method is used to purify the donor cells. After the tissues was disassociated, an adhesion step was performed to remove any possible contaminating adherent cells followed by a density gradient purification step. The majority of lymphocytes are quiescent (in G0 phase) and therefore can have a methylation status than conveys greater plasticity for reprogramming. Multipotent or pluripotent stem cells or cell lines useful as donor cells in embodiments of the present invention are functionally defined as stem cells by their ability to undergo differentiation into a variety of cell types including, but not limited to, adipogenic, neurogenic, osteogenic, chondrogenic and cardiogenic cell.

In some embodiments, host cell enucleation for the generation of hybrid stem cells according to the teachings of the present invention can be conducted using a variety of means. In a non-limiting example, ADSCs were plated onto fibronectin coated tissue culture slides and treated with cells with either cytochalasin D or cytochalasin B. After treatment, the cells can be trypsinized, re-plated and are viable for about 72 hours post enucleation. Host cells and donor nuclei can be fused using one of a number of fusion methods known to those of skill in the art, including but not limited to electrofusion, microinjection, chemical fusion or virus-based fusion, and all methods of cellular fusion are envisioned as being within the scope of the present invention. The hybrid stem cells made according to the teachings of the present invention possess surface antigens and receptors from the enucleated host cell but has a nucleus from a developmentally younger cell. Consequently, the hybrid stem cells of the present invention will be receptive to cytokines, chemokines and other cell signaling agents, yet possess a nucleus free from age-related DNA damage. The therapeutically reprogrammed cells and hybrid stem cells made in accordance with the teachings of the present invention are useful in a wide range of therapeutic applications for cellular regenerative/reparative therapy. For example, and not intended as a limitation, the therapeutically reprogrammed cells and hybrid stem cells of the present invention can be used to replenish stem cells in animals whose natural stem cells have been depleted due to age or ablation therapy such as cancer radiotherapy and chemotherapy. In another non-limiting example, the therapeutically reprogrammed cells and hybrid stem cells of the present invention are useful in organ regeneration and tissue repair. In one embodiment of the present invention, therapeutically reprogrammed cells and hybrid stem cells can be used to reinvigorate damaged muscle tissue including dystrophic muscles and muscles damaged by ischemic events such as myocardial infarcts. In another embodiment of the present invention, the therapeutically reprogrammed cells and hybrid stem cells disclosed herein can be used to ameliorate scarring in animals, including humans, following a traumatic injury or surgery. In this embodiment, the therapeutically reprogrammed cells and hybrid stem cells of the present invention are administered systemically, such as intravenously, and migrate to the site of the freshly traumatized tissue recruited by circulating cytokines secreted by the damaged cells. In another embodiment of the present invention, the therapeutically reprogrammed cells and hybrid stem cells can be administered locally to a treatment site in need of repair or regeneration.

In one embodiment, umbilical cord samples were obtained following the delivery of normal term babies with Institutional Review Board approval. A portion of the umbilical cord was then cut into approximately 3 cm long segments. The segments were then placed immediately into 25 ml of phosphate buffered saline without calcium and magnesium (PBS) and 1.times. antibiotics (100 U/ml penicillin, 100 ug/ml streptomycin, 0.025 ug/ml amphotericin B). The tubes were then brought to the lab for dissection within 6 hours. Each 3 cm umbilical cord segment was dissected longitudinally utilizing aseptic technique. The tissue was carefully undermined and the umbilical vein and both umbilical arteries were removed. The remaining segment was sutured inside out and incubated in 25 ml of PBS, 1.times. antibiotic, and 1 mg/ml of collagenase at room temperature. After 16-18 hours the remaining suture and connective tissue was removed and discarded. The cell suspension was separated equally into two tubes, the cells were washed 3.times. by diluting with PBS to yield a final volume of 50 ml per tube, and then centrifuged. Red blood cells were then lysed using a hypotonic solution. Cells were plated onto 6-well plates at a concentration of 5-20.times.10.sup.6 cells per well. UC-MSC were cultured in low-glucose DMEM (Gibco) with 10% FBS (Hyclone), 2 mM L-Glutamine (Gibco), 100 U/ml penicillin, 100 ug/ml streptomycin, 0.025 ug/ml amphotericin B (Gibco). Cells were washed 48 hours after the initial plating with PBS and given fresh media. Cell culture media were subsequently changed twice a week through half media changes. After 7 days or approximately 70-80% confluence, cells were passed using HyQTase (Hyclone) into a 10 cm plate. Cells were then regularly passed 1:2 every 7 days or upon reaching 80% confluence. Alternatively, 0.25% HQ trypsin/EDTA (Hyclone) was used to passage cells in a similar manner.

In some embodiments of the invention, administration of cells of the invention is performed for suppression of an inflammatory and/or autoimmune disease. In these situations, it may be necessary to utilize an immune suppressive/or therapeutic adjuvant. Immune suppressants are known in the art and can be selected from a group comprising of: cyclosporine, rapamycin, campath-1H, ATG, Prograf, anti IL-2r, MMF, FTY, LEA, cyclosporin A, diftitox, denileukin, levamisole, azathioprine, brequinar, gusperimus, 6-mercaptopurine, mizoribine, rapamycin, tacrolimus (FK-506), folic acid analogs (e.g., denopterin, edatrexate, methotrexate, piritrexim, pteropterin, Tomudex®, and trimetrexate), purine analogs (e.g., cladribine, fludarabine, 6-mercaptopurine, thiamiprine, and thiaguanine), pyrimidine analogs (e.g., ancitabine, azacitidine, 6-azauridine, carmofur, cytarabine, doxifluridine, emitefur, enocitabine, floxuridine, fluorouracil, gemcitabine, and tegafur) fluocinolone, triaminolone, anecortave acetate, fluorometholone, medrysone, prednislone, etc. In another embodiment, the use of stem cell conditioned media may be used to potentiate an existing anti-inflammatory agent. Anti-inflammatory agents may comprise one or more agents including NSAIDs, interleukin-1 antagonists, dihydroorotate synthase inhibitors, p38 MAP kinase inhibitors, TNF-α inhibitors, TNF-α sequestration agents, and methotrexate.

An embodiment of the present disclosure provides a modified mesenchymal stem cell culture medium, comprising a basal culture medium, a first component, and melatonin, wherein the first component is at least one selected from the group consisting of coenzyme Q10 and mitoquinone mesylate, and has a working concentration in a range from 1 .mu.M to 20 .mu.M, the melatonin has a working concentration in a range from 1 .mu.M to 20 .mu.M, and the first component and the melatonin are in a molar ratio ranging from 1:0.2 to 1:10. The modified mesenchymal stem cell culture medium can delay the decrease of mitochondrial activity in mesenchymal stem cells and increase the number of mitochondria. The mesenchymal stem cells cultured in this culture medium have high mitochondrial activity and a large number of mitochondria. Specifically, the basal culture medium is used to provide essential nutrients for the growth of the mesenchymal stem cells. The basal culture medium in the present disclosure is one selected from DMEM, EMEM, IMDM, GMEM, RPMI-1640, and .alpha.-MEM. The basic culture medium may be a serum-free mesenchymal stem cell culture medium, or a serum-containing mesenchymal stem cell culture medium. In an embodiment, the modified mesenchymal stem cell culture medium contains an additive. Specifically, the additive may be well-known additives as long as they do not inhibit the proliferation of the mesenchymal stem cells. There are, for example, growth factors (such as insulin, etc.), iron sources (such as transferrin, etc.), polyamines (such as putrescine, etc.), minerals (such as sodium selenate, etc.), sugars (such as glucose, etc.), organic acids (such as pyruvic acid, lactic acid, etc.), or the like. In an embodiment, the first component is coenzyme Q10. In an embodiment, the first component is mitoquinone mesylate (mitoQ for short). In an embodiment, the working concentration of the first component is in a range from 1 .mu.M to 20 .mu.M. The first component at the working concentration in a range from 1 .mu.M to 20 .mu.M may protect the mitochondira, increase the mitochondrial activity, and reduce ROS. Further, the working concentration of the first component is in a range from 1 .mu.M to 10 .mu.M. Still further, the working concentration of the first component is in a range from 1 .mu.M to 5 .mu.M. In an embodiment, the working concentration of the coenzyme Q10 is in a range from 1 .mu.M to 20 .mu.M. The coenzyme Q10 at the working concentration in a range from 1 .mu.M to 20 .mu.M may protect the mitochondira, increase the mitochondrial activity, and reduce ROS. Further, the working concentration of the coenzyme Q10 is in a range from 1 .mu.M to 10 .mu.M. Still further, the working concentration of the coenzyme Q10 is in a range from 1 .mu.M to 5 .mu.M.

In an embodiment, the first component and the melatonin are in a molar ratio ranging from 1:0.2 to 1:10. The first component and the melatonin in a molar ratio ranging from 1:0.2 to 1:10 may promote the proliferation of mesenchymal stem cells, enhance the cell viability, increase the mitochondrial activity, and reduce ROS. Further, the first component and the melatonin are in a molar ratio ranging from 1:1 to 1:3. In an embodiment, the first component is coenzyme Q10, and the first component and the melatonin are in a molar ratio ranging from 1:0.2 to 1:10, and further, in a molar ratio ranging from 1:1 to 1:3.In an embodiment, the working concentration of the melatonin is in a range from 1 .mu.M to 20 .mu.M. Further, the working concentration of the melatonin is in a range from 1 .mu.M to 10 .mu.M. Still further, the working concentration of the melatonin Q10 is in a range from 1 .mu.M to 5 .mu.M. In an embodiment, the modified mesenchymal stem cell culture medium further comprises a second component, and the second component is at least one selected from the group consisting of nicotinamide mononucleotide (NMN), nicotinamide ribose (NR), and NADH. The second component functions together with the first component and melatonin to further increase the mitochondrial activity and the number of mitochondria in mesenchymal stem cells. Further, the first component and the second component are in a molar ratio ranging from 1:0.2 to 1:10. When the first component and the second component are in a molar ratio ranging from 1:0.2 to 1:10, the mitochondrial activity can be further increased. Still further, the first component and the second component are in a molar ratio ranging from 1:0.2 to 1:2.5. In an embodiment, the second component is the nicotinamide mononucleotide, and the nicotinamide mononucleotide has a working concentration in a range from 0.5 .mu.M to 10 .mu.M. Further, the working concentration of the nicotinamide mononucleotide is in a range from 0.5 .mu.M to 5 .mu.M. In an embodiment, the first component is coenzyme Q10, the coenzyme Q10 has a working concentration in a range from 1 .mu.M to 5 .mu.M, the working concentration of the melatonin is in a range from 1 .mu.M to 5 .mu.M, and the working concentration of the nicotinamide mononucleotide is in a range from 0.5 .mu.M to 10 .mu.M. Further, the coenzyme Q10 and the melatonin are in a molar ratio from 1:1 to 1:3; the coenzyme Q10 and the nicotinamide mononucleotide are in a molar ratio from 1:0.2 to 1:2.5. In an embodiment, the modified mesenchymal stem cell culture medium is composed of a basal culture medium, a serum substitute, EGF, BEGF, coenzyme Q10, melatonin, and nicotinamide mononucleotide; wherein the working concentration of the coenzyme Q10 is in a range from 1 .mu.M to 5 .mu.M, the working concentration of the melatonin is in a range from 1 .mu.M to 5 .mu.M, and the working concentration of the nicotinamide mononucleotide is in a range from 0.5 .mu.M to 10 .mu.M. Further, the coenzyme Q10 and the melatonin are in a molar ratio from 1:1 to 1:3; the coenzyme Q10 and the nicotinamide mononucleotide are in a molar ratio from 1:0.2 to 1:2.5. In an embodiment, the modified mesenchymal stem cell culture medium further comprises at least one of vitamin B, minerals (or inorganic salt), polyphenols, L-carnitine, alpha lipoic acid, pyrroloquinoline quinone, and creatine. It should be noted that the working concentration of the first component refers to a concentration of the first component in the modified mesenchymal stem cell culture medium; the working concentration of melatonin refers to a concentration of the melatonin in the modified mesenchymal stem cell culture medium; and the working concentration of the second component refers to a concentration of the second component in the modified mesenchymal stem cell culture medium. In an embodiment, the modified mesenchymal stem cell culture medium as described above is used to culture the bone marrow mesenchymal stem cells. In an embodiment, the modified mesenchymal stem cell culture medium as described above can also be used to culture other mesenchymal stem cells, for example, umbilical cord mesenchymal stem cells, other than the bone marrow mesenchymal stem cells, and may increase the activity and number of mitochondria of other mesenchymal stem cells. An embodiment of the present disclosure also provides a method for culturing the bone marrow mesenchymal stem cells, comprising step a) and b), specifically, step a): isolating bone marrow mesenchymal stem cells from bone marrow. Specifically, the method for isolating bone marrow mesenchymal stem cells from bone marrow is a whole bone marrow culture method (also called a direct culture method) or a density gradient centrifugation method. In an embodiment, the bone marrow mesenchymal stem cells are isolated from the bone marrow by the density gradient centrifugation method, specifically, by adding normal saline in an equal volume to the bone marrow for dilution, followed by the diluted bone marrow slowly onto a surface of lymphocyte isolation liquid (Ficoll isolation liquid) in a ratio of 1 part by volume of Ficoll isolation liquid to 2 parts by volume of the diluted bone marrow, centrifuging the resultant mixture in a horizontal centrifuge at 2000 r/min at 20.degree. C. for 25-30 minutes, and removing the bone marrow mesenchymal stem cells from the interface. Then, the bone marrow mesenchymal stem cells are washed with normal saline 2 to 3 times and counted after adding an appropriate amount of culture medium. These cells, especially when treated with hypoxia are useful for treatment of ovarian and/or endometrial failure. In this embodiment, the bone marrow is derived from human. It is understood that in some other embodiments, the bone marrow may also be animal bone marrow, such as mouse bone marrow. In some embodiments, step a) may be omitted, and the bone marrow mesenchymal stem cells may be purchased.

In an embodiment, the step of primarily culturing the bone marrow mesenchymal stem cells includes: seeding the bone marrow mesenchymal stem cells isolated from bone marrow at a concentration of 5.times.10.sup.6 cells/mL in any of the modified mesenchymal stem cell culture medium as described above for culture, refreshing the medium after 3 days to remove non-adherent cells, and refreshing half of the spent medium every 3 to 4 days. It should be noted that the primary culture herein refers to the culture during the period from seeding the cells obtained from the tissue taken out from human's body to the first subculture. In an embodiment, a subculture step is also included after the step of primarily culturing the bone marrow mesenchymal stem cells. Specifically, the subculture step is performed when the cells reach 90% confluency. Further, the subculture step includes: digesting the bone marrow mesenchymal stem cells, and seeding the digested bone marrow mesenchymal stem cells at a density of 2.times.10.sup.5 cells/mL to 5.times.10.sup.5 cells/mL in a fresh modified mesenchymal stem cell medium for culture.

Administration of mesenchymal stem cell exosomes may be performed into the ovary directly as a means of treating ovarian and/or endometrial failure. A “mesenchymal stem cell (MSC)” is a progenitor cell having the capacity to differentiate into neuronal cells, adipocytes, chondrocytes, osteoblasts, myocytes, cardiac tissue, and other endothelial or epithelial cells. These cells may be defined phenotypically by gene or protein expression. These cells have been characterized to express (and thus be positive for) one or more of CD13, CD29, CD44, CD49a, b, c, e, f, CD51, CD54, CD58, CD71, CD73, CD90, CD102, CD105, CD106, CDw119, CD120a, CD120b, CD123, CD124, CD126, CD127, CD140a, CD166, P75, TGF-bIR, TGF-bIIR, HLA-A, B, C, SSEA-3, SSEA-4, D7 and PD-L1. These cells have also been characterized as not expressing (and thus being negative for) CD3, CD5, CD6, CD9, CD10, CD11a, CD14, CD15, CD18, CD21, CD25, CD31, CD34, CD36, CD38, CD45, CD49d, LD50, CD62E, L, S, CD80, CD86, CD95, CD117, CD133, SSEA-1, and ABO. Thus, MSCs may be characterized phenotypically and/or functionally according to their differentiation potential.

Commercially available media may be used for the growth, culture and maintenance of MSCs. Such media include but are not limited to Dulbecco's modified Eagle's medium (DMEM). Components in such media that are useful for the growth, culture and maintenance of MSCs, fibroblasts, and macrophages include but are not limited to amino acids, vitamins, a carbon source (natural and non-natural), salts, sugars, plant derived hydrolysates, sodium pyruvate, surfactants, ammonia, lipids, hormones or growth factors, buffers, non-natural amino acids, sugar precursors, indicators, nucleosides and/or nucleotides, butyrate or organics, DMSO, animal derived products, gene inducers, non-natural sugars, regulators of intracellular pH, betaine or osmoprotectant, trace elements, minerals, non-natural vitamins. Additional components that can be used to supplement a commercially available tissue culture medium include, for example, animal serum (e.g., fetal bovine serum (FBS), fetal calf serum (FCS), horse serum (HS)), antibiotics (e.g., including but not limited to, penicillin, streptomycin, neomycin sulfate, amphotericin B, blasticidin, chloramphenicol, amoxicillin, bacitracin, bleomycin, cephalosporin, chlortetracycline, zeocin, and puromycin), and glutamine (e.g., L-glutamine). Mesenchymal stem cell survival and growth also depends on the maintenance of an appropriate aerobic environment, pH, and temperature. MSCs can be maintained using methods known in the art, e.g., as described in Pittenger et al., Science, 284:143-147 (1999), incorporated herein by reference.

In some embodiments, the MSC exosomes used to treat the ovaries are isolated. As used herein, an “isolated exosome” is an exosome that is physically separated from its natural environment. An isolated exosome may be physically separated, in whole or in part, from tissue or cells with which it naturally exists (e.g., MSCs). In some embodiments, the isolated MSC exosomes are isolated from the culturing media of MSCs from human bone marrow, umbilical cord Wharton's Jelly, or adipose tissue. Such culturing media is termed “MSC-conditioned media” herein. In some embodiments, isolated exosomes may be free of cells such as MSCs, or it may be free or substantially free of conditioned media, or it may be free of any biological contaminants such as proteins. Typically, the isolated exosomes are provided at a higher concentration than exosomes present in un-manipulated conditioned media. In some embodiments, exosomes are collected from mesenchymal stem cells that have been stimulated.

In one embodiment, activated, or “stimulated” mesenchymal stem cells are treated with conditions such as “hypoxia”, exposed to inflammatory stimuli such as cytokines and/or toll like receptor agonists. In some embodiments, the isolated MSC exosome described herein comprises one or more (e.g., 1, 2, 3, 4, 5, or more) known exosome markers. In some embodiments, the known exosome markers are selected from the group consisting of: FLOT1 (Flotillin-1, Uniprot ID: 075955), CD9 (CD9 antigen, Uniprot ID: P21926), and CD63 (CD63 antigen, Uniprot ID: P08962). In some embodiments, the isolated MSC exosome is substantially free of contaminants (e.g., protein contaminants). The isolated MSC exosome is “substantially free of contaminants” when the preparation of the isolated MSC exosome contains fewer than 20%, 15%, 10%, 5%, 2%, 1%, or less than 1%, of any other substances (e.g., proteins). In some embodiments, the isolated MSC is “substantially free of contaminants” when the preparation of the isolated MSC exosome is at least 80%, at least 85%, at least 90%, at least 95%, at least 98%, at least 99%, at least 99.9% pure, with respect to contaminants (e.g., proteins). “Protein contaminants” refer to proteins that are not associated with the isolated exosome and do not contribute to the biological activity of the exosome. The protein contaminants are also referred to herein as “non-exosomal protein contaminants.” In some embodiments, the isolated MSC exosome used in accordance with the present disclosure has a diameter of about 30-150 nm. For example, the isolated MSC exosome may have a diameter of 30-150 nm, 30-140 nm, 30-130 nm, 30-120 nm, 30-110 nm, 30-100 nm, 30-90 nm, 30-80 nm, 30-70 nm, 30-60 nm, 30-50 nm, 30-40 nm, 40-150 nm, 40-140 nm, 40-130 nm, 40-120 nm, 40-110 nm, 40-100 nm, 40-90 nm, 40-80 nm, 40-70 nm, 40-60 nm, 40-50 nm, 50-150 nm, 50-140 nm, 50-130 nm, 50-120 nm, 50-110 nm, 50-100 nm, 50-90 nm, 50-80 nm, 50-70 nm, 50-60 nm, 60-150 nm, 60-140 nm, 60-130 nm, 60-120 nm, 60-110 nm, 60-100 nm, 60-90 nm, 60-80 nm, 60-70 nm, 70-150 nm, 70-140 nm, 70-130 nm, 70-120 nm, 70-110 nm, 70-100 nm, 70-90 nm, 70-80 nm, 80-150 nm, 80-140 nm, 80-130 nm, 80-120 nm, 80-110 nm, 80-100 nm, 80-90 nm, 90-150 nm, 90-140 nm, 90-130 nm, 90-120 nm, 90-110 nm, 90-100 nm, 100-150 nm, 100-140 nm, 100-130 nm, 100-120 nm, 100-110 nm, 110-150 nm, 110-140 nm, 110-130 nm, 110-120 nm, 120-150 nm, 120-140 nm, 120-130 nm, 130-150 nm, 130-140 nm, or 140-150 nm. In some embodiments, the isolated MSC exosome may have a diameter of about 50 nm, 60 nm, 70 nm, 80 nm, 90 nm, 100 nm, 110 nm, 120 nm, 130 nm, 140 nm, or 150 nm. In some embodiments, the isolated MSC exosomes exhibit a biconcave morphology. As described herein, the isolated MSC exosomes can be used to treat the monocytes to modulate the monocyte phenotype (e.g., both in vitro and in vivo such as in the bone marrow). “Treat a monocyte with an isolated MSC exosome” means contacting the monocyte with a MSC exosome (e.g., for a period of time). In some embodiments, the treating (i.e., contacting) is carried out in vitro. For example, monocytes may be cultured in vitro and isolated MSC exosomes may be added to the culture such that the monocytes contact the isolated MSC exosomes. In some embodiments, the treating (i.e., contacting) is carried out ex vivo. For example, monocytes may be isolated from the bone marrow of a subject and isolated MSC exosomes may be added to the monocytes such that the monocytes contact the isolated MSC exosomes. In some embodiments, the treating (i.e., contacting) is carried out in vivo. For example, the isolated MSC exosomes may be administered to a subject (e.g., via intravenous injection), reach the one marrow, and contact the monocytes in the bone marrow. In some embodiments, the monocyte is treated (i.e., contacted) with the MSC exosome for at least 1 hour (e.g., at least 1, at least 2, at least 3, at least 4, at least 5, at least 6, a least 7, at least 8, at least 9, at least 10, at least 15, at least 20, at least 25, at least 30, at least 35, at least 40, at least 45, at least 50, at least 55, at least 60, at least 65, at least 70, at least 75, at least 80, at least 85, at least 90, at least 95, at least 100 hours, or longer). In some embodiments, the monocyte is treated (i.e., contacted) with the MSC exosome for 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 hours, or longer. In some embodiments, the monocyte has been polarized to a pro-inflammatory state as a result of environmentally or developmentally-precipitated injury, and its polarity is modulated to a regulatory phenotype upon contact with the isolated MSC exosome. In some embodiments, the monocyte is a pro-inflammatory monocyte prior to being treated (i.e., contacted) with the isolated MSC exosome, and is a regulatory monocyte after being treated (i.e., contacted) with the isolated MSC exosome. In some embodiments, a mixture of pro-inflammatory monocytes and regulatory monocytes are contacted with isolated MSC exosomes and the treating results in a higher ratio (e.g., at least 10% higher) of regulatory monocytes in the mixture, being treated with isolated MSC exosomes. For example, the ratio of regulatory monocytes may be at least 10%, at least 20%, at least 30%, at least 40%, at least 50%, at least 60%, at least 70%, at least 80%, at least 90%, at least 100%, at least 2-fold, at least 5-fold, at least 10-fold, at least 100-fold, or higher after being treated with MSC exosomes, compared to before being treated with isolated MSC exosomes. In some embodiments, the ratio of regulatory monocytes is 10%, 20%, 30%, 40%, 50%, 60%, 70%, 80%, 90%, 100%, 2-fold, 5-fold, 10-fold, 100-fold, or higher after being treated with MSC exosomes, compared to before being treated with isolated MSC exosomes. Further provided herein are uses of the monocytes treated with isolated MSC exosomes for treating a disease (e.g., a fibrotic disease such as pulmonary fibrosis or an autoimmune disease). In some embodiments, the monocytes treated with isolated MSC exosomes are used in the manufacturing of a medicament for treating a disease (e.g., a fibrotic disease or an autoimmune disease). Compositions comprising monocytes treated with isolated MSC exosomes are also provided. In some embodiments, the monocytes treated with isolated MSC exosomes are formulated in a composition for the treatment of ovarian and/or endometrial failure. In some embodiments, the composition comprising monocytes treated with isolated MSC exosomes further comprises a second agent.

1. A method for treatment of infertility comprising the steps of: a) obtaining a patient suffering from infertility, wherein said cause of infertility is associated with abnormalities of hormone production and/or responsiveness to said hormones; b) administering a regenerative cell population in a manner to stimulate ovarian functionality; c) administering a regenerative cell population in a manner to stimulate ovarian functionality; d) providing prior to and/or concurrently with, and/or subsequently to, an agent or plurality of agents which alter the host in order to enhance ability of administered regenerative cells to provide a therapeutic benefit; and e) optionally providing a mitogenic and/or stem cell activator agent.

2. The method of claim 1, wherein said mesenchymal stem cells are derived from tissues.

3. The method of claim 2, wherein said tissue derived mesenchymal stem cells are selected from the group consisting of: a) bone marrow; b) perivascular tissue; c) adipose tissue; d) placental tissue; e) amniotic membrane; f) omentum; g) tooth; h) umbilical cord tissue; i) fallopian tube tissue; j) hepatic tissue; k) renal tissue; l) cardiac tissue; m) tonsillar tissue; n) testicular tissue; o) ovarian tissue; p) neuronal tissue; q) auricular tissue; r) colonic tissue; s) submucosal tissue; t) hair follicle tissue; u) pancreatic tissue; v) skeletal muscle tissue; and w) subepithelial umbilical cord tissue.

4. The method of claim 3, wherein said tissue derived mesenchymal stem cells are isolated from tissues containing cells selected from the group of cells consisting of: endothelial cells, epithelial cells, dermal cells, endodermal cells, mesodermal cells, fibroblasts, osteocytes, chondrocytes, natural killer cells, dendritic cells, hepatic cells, pancreatic cells, stromal cells, and salivary gland mucous cells.

5. The method of claim 1, in which the patient is prepared for administration of regenerative cells by evoking a regenerative state in the ovary and/or endometrium.

6. The method of claim 5, wherein said regenerative state is evoked by administration of extracorporeal shock waves.

7. The method of claim 6, wherein said extracorporeal shock wave generates a flux density higher than 0.3 mJ/mm.sup.2.

8. The method of claim 6, wherein said extracorporeal shock wave is administered at an energy of 1-1000 joules.

9. The method of claim one wherein stimulation of ovarian function in a patient suffering from ovarian failure further comprises the steps of: a) obtaining peripheral blood; b) isolating platelet rich plasma, and/or platelet lysate (fresh, frozen and lyophilized); c) quantifying growth factor content of said platelet rich plasma and/or platelet lysate; d) optionally concentrating said growth factors from said platelet rich plasma and/or platelet lysate derived growth factors; and e) administering said growth factors locally into ovarian tissue in a patient in need of treatment.

10. The method of claim 9, wherein said growth factor associated with platelet rich plasma and/or platelet lysate is EGF-1.

11. The method of claim 9, wherein said growth factor associated with platelet rich plasma and/or platelet lysate is IGF-1.

12. The method of claim 9, wherein said growth factor associated with platelet rich plasma and/or platelet lysate is activin.

13. The method of claim 9, wherein said growth factor associated with platelet rich plasma and/or platelet lysate is angiopoietin.

14. The method of claim 9, wherein said growth factors are exosomes. The method of claim 9, wherein said growth factors are microvesicles.

16. The method of claim 1, wherein increasing fertility is achieved through the steps comprising of: a) extracting an autologous population of regenerative cells; b) treating said autologous population of regenerative cells with platelet rich plasma, and/or platelet lysate at a concentration and duration sufficient to induce type 2 cytokine polarization; and c) administering said treated regenerative cells into a patient in need of treatment.

17. The method of claim 16, wherein said autologous regenerative cells are bone marrow mononuclear cells/aspirate.

18. The method of claim 1, wherein agents possessing anti-inflammatory properties are selected from the group consisting of: anti-TNF-α, lysophylline, alpha 1-antitrypsin (AAT), interleukin-10 (IL-10), pentoxyfilline, COX-2 inhibitors, 21-acetoxypregnenolone, alclometasone, algestone, amcinonide, beclomethasone, betamethasone, budesonide, chloroprednisone, clobetasol, clobetasone, clocortolone, cloprednol, corticosterone, cortisone, cortivazol, deflazacort, desonide, desoximetasone, dexamethasone, diflorasone, diflucortolone, difluprednate, enoxolone, fluazacort, flucloronide, flumethasone, flunisolide, fluocinolone acetonide, fluocinonide, fluocortin butyl, fluocortolone, fluorometholone, fluperolone acetate, fluprednidene acetate, fluprednisolone, flurandrenolide, fluticasone propionate, formocortal, halcinonide, halobetasol propionate, halometasone, halopredone acetate, hydrocortamate, hydrocortisone, loteprednol etabonate, mazipredone, medrysone, meprednisone, methylprednisolone, mometasone furoate, paramethasone, prednicarbate, prednisolone, prednisolone 25-diethylamino-acetate, prednisolone sodium phosphate, prednisone, prednival, prednylidene, rimexolone, tixocortol, triamcinolone, triamcinolone acetonide, triamcinolone benetonide, triamcinolone hexacetonide, aminoarylcarboxylic acid derivatives (e.g., enfenamic acid, etofenamate, flufenamic acid, isonixin, meclofenamic acid, mefenamic acid, niflumic acid, talniflumate, terofenamate, tolfenamic acid), arylacetic acid derivatives (e.g., aceclofenac, acemetacin, alclofenac, amfenac, amtolmetin guacil, bromfenac, bufexamac, cinmetacin, clopirac, diclofenac sodium, etodolac, felbinac, fenclozic acid, fentiazac, glucametacin, ibufenac, indomethacin, isofezolac, isoxepac, lonazolac, metiazinic acid, mofezolac, oxametacine, pirazolac, proglumetacin, sulindac, tiaramide, tolmetin, tropesin, zomepirac), arylbutyric acid derivatives (e.g., bumadizon, butibufen, fenbufen, xenbucin), arylcarboxylic acids (e.g., clidanac, ketorolac, tinoridine), arylpropionic acid derivatives (eg., alminoprofen, benoxaprofen, bermoprofen, bucloxic acid, carprofen, fenoprofen, flunoxaprofen, flurbiprofen, ibuprofen, ibuproxam, indoprofen, ketoprofen, loxoprofen, naproxen, oxaprozin, piketoprolen, pirprofen, pranoprofen, protizinic acid, suprofen, tiaprofenic acid, ximoprofen, zaltoprofen), pyrazoles (e.g., difenamizole, epirizole), pyrazolones (e.g., apazone, benzpiperylon, feprazone, mofebutazone, morazone, oxyphenbutazone, phenylbutazone, pipebuzone, propyphenazone, ramifenazone, suxibuzone, thiazolinobutazone), salicylic acid derivatives (e.g., acetaminosalol, aspirin, benorylate, bromosaligenin, calcium acetylsalicylate, diflunisal, etersalate, fendosal, gentisic acid, glycol salicylate, imidazole salicylate, lysine acetylsalicylate, mesalamine, morpholine salicylate, 1-naphthyl salicylate, olsalazine, parsalmide, phenyl acetylsalicylate, phenyl salicylate, salacetamide, salicylamide o-acetic acid, salicylsulfuric acid, salsalate, sulfasalazine), thiazinecarboxamides (e.g., ampiroxicam, droxicam, isoxicam, lornoxicam, piroxicam, tenoxicam), epsilon.-acetamidocaproic acid, s-adenosylmethionine, 3-amino-4-hydroxybutyric .acid, amixetrine, bendazac, benzydamine, α-bisabolol, bucolome, difenpiramide, ditazol, emorfazone, fepradinol, guaiazulene, nabumetone, nimesulide, oxaceprol, paranyline, perisoxal, proquazone, superoxide dismutase, tenidap, zileuton, candelilla wax, alpha bisabolol, aloe vera, Manjistha, Guggal, kola extract, chamomile, sea whip extract, glycyrrhetic acid, glycyrrhizic acid, oil soluble licorice extract, monoammonium glycyrrhizinate, monopotassium glycyrrhizinate, dipotassium glycyrrhizinate, 1-beta-glycyrrhetic acid, stearyl glycyrrhetinate, and 3-stearyloxy-glycyrrhetinic acid.

19. The method of claim 1, wherein the autologous regenerative cell is combined with an anti-oxidant selected from the group consisting of: beta carotene, Vitamin E, anthocyanins, selenium, catechins, lutein, lycopene, n-acetylcysteine, ascorbic acid, glutathione, vitamin k3, resveratrol, a lipoic acid, quercetin, kaempferol, myricetin, apigenin, luteolin, curcumin, and caffeic acid in order to increase therapeutic benefit.

20. The method of claim 1, wherein the autologous regenerative cell is combined with a fertility hormone selected from the group consisting of: Gonal-F, Follistim, Bravelle, Menopur, Repronex, Estrace, Clomid, serophene, follicle stimulation hormone, luteinizing hormone, human chorionic gonadotrophin, estrogen, GnRH antagonist, progesterone, gonadotropin-releasing hormone, Human menopausal gonadotropin, Metrodin, Pergonal, Factrel, Lutrepulse, Lupron, Synarel, Zoladex, Antagon, Cetrotide, Novarel, Ovidrel, Pregnyl, Profasi, Antagon, Dostinex, Parlodel, and Fertinex.
